(19) United States
(12) Patent Application Publication
(10) Pub. No.: US 2016/0158251 A1
(43) Pub. Date: Jun. 9, 2016

(54) COMPOSITION FOR PREVENTING OR TREATING MUSCULAR ATROPHY OR PROMOTING MUSCULAR REGENERATION IN SUBJECT COMPRISING SULFONAMIDE COMPOUND AND USE THEREOF

(71) Applicant: Samsung Electronics Co., Ltd., Suwon-si (KR)

(72) Inventors: Euiseok Shin, Yongin-si (KR); Hyeonju Yeo, Seoul (KR); Jumi Kim, Yongin-si (KR); Sungchun Cho, Hwaseongi-si (KR); Sangchul Park, Seongnam-si (KR)

(21) Appl. No.: 14/812,705

(22) Filed: Jul. 29, 2015

(30) Foreign Application Priority Data
Dec. 9, 2014 (KR) 10-2014-0175873

(51) Int. Cl. A61K 31/635; A61K 31/63

(52) U.S. Cl. CPC A61K 31/635; A61K 31/63

(57) ABSTRACT

A composition for preventing or treating muscular atrophy or promoting muscular regeneration in a subject including a sulfonamide compound, a pharmaceutically acceptable salt, solvate, or polymorph thereof, or a combination of at least two of the foregoing, and a method of preventing or treating muscular atrophy or promoting muscular regeneration by using the composition.

### RELATED APPLICATION

This application claims the benefit of Korean Patent Application No. 10-2014-0175873, filed on Dec. 9, 2014, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 1,630 byte ASCII (Text) file named “720627_ST25.TXT” created Jul. 22, 2015.

### BACKGROUND

1. Field

The present disclosure relates to compositions for preventing or treating muscular atrophy or promoting muscular regeneration in a subject including a sulfonamide compound, and methods employing the compositions.

2. Description of the Related Art

Muscle is tissue that is essential for human movement and performance. When the amount of proteins constituting muscle fibers decreases or loss of muscle fibers increases under various pathological conditions, muscle mass decreases and the decreased muscle mass causes muscular atrophy. Muscle mass may decrease by aging, dystrophy, non-use of muscles, damaged nerves, and use of glucocorticoid hormones. Under pathological conditions causing damage to muscles, satellite cells, which are stem cells differentiating into muscle cells for muscular regeneration, are activated. The activated satellite cells proliferate, differentiate, and fuse into existing muscle fiber cells or muscle cells to constitute new muscle fibers, thereby restoring damaged muscles. Thus, muscular atrophy may be prevented or treated by promoting muscular regeneration and restoring functions of muscles by promoting proliferation of skeletal muscle satellite cells.

Also, glucocorticoid hormones administered to a body or overproduced in a body (e.g., Cushing's syndrome) may cause loss of muscle mass. One of the side effects of treatment with glucocorticoid hormone formulations is decreased muscle mass. Research indicates that glucocorticoid hormone formulations activate a signal transmission process mediated by a glucocorticoid receptor, thereby increasing the expression of ubiquitin ligases, such as Atrogin-1 or MuRF-1 protein, which degrade muscle proteins. These ubiquitin ligases degrade proteins essential for synthesis of muscles, such as MyoD or myosin heavy chains, thereby causing loss of muscle mass.

Thus, there is still a need to develop methods of preventing or treating muscular atrophy by promoting proliferation of skeletal muscle satellite cells and inhibiting loss of muscle mass.

### SUMMARY

Provided is a method of preventing or treating muscular atrophy in a subject including administering a sulfonamide compound, a pharmaceutically acceptable salt, solvate, or polymorph thereof, or a combination of at least two of the foregoing to the subject.

Provided is a method of promoting muscular regeneration in a subject including administering a sulfonamide compound, a pharmaceutically acceptable salt, solvate, or polymorph thereof, or a combination of at least two of the foregoing to the subject.

Additional aspects will be set forth in part in the description which follows and, in part, will be apparent from the description, or may be learned by practice of the presented exemplary embodiments.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a graph illustrating the fold increase (multiples) in proliferation of skeletal muscle satellite cells using 0.1 μM, 1 μM, and 10 μM of each of sulfadiazine, sulfamethoxazole, sulfadoxine, and sulfaguanidine (*: p<0.05);

FIG. 2A is a graph illustrating relative expression levels (arbitrary unit (AU)) of mRNA of Atrogin-1 when C2C12 myoblast cells were cultured in the presence of 1 μM, 10 μM, or 100 μM of sulfadiazine (*: p<0.05 vs. dexamethasone);

FIG. 2B is a graph illustrating relative expression levels (arbitrary unit (AU)) of mRNA of MURF-1 when C2C12 myoblast cells were cultured in the presence of 1 μM, 10 μM, or 100 μM of sulfadiazine (*: p<0.05 vs. dexamethasone); and

FIG. 3 is a graph illustrating diameters (μm) of muscle fibers when C2C12 myoblast cells were cultured in the presence of dexamethasone, or in the presence of dexamethasone and 100 μM of sulfadiazine (*: p<0.05).

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Provided is a pharmaceutical composition for preventing or treating muscular atrophy in a subject includes a compound represented by Formula 1 below, a pharmaceutically acceptable salt, solvate, or polymorph thereof, or a combination of at least two of the foregoing.

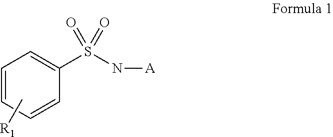

The compound represented by Formula 1 is a sulfonamide compound. Optionally, group A is double bonded to the nitrogen (N) shown in the above Formula. When group A is single bonded to the nitrogen, the nitrogen may be also be bound to hydrogen (e.g., —NH—). Thus, Formula 1 may be presented as:

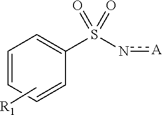

wherein the dotted line is an optional double bond. The sulfonamide compound is used as a generic name that covers all forms in which a hydroxyl group of a sulfonic acid (—SO₃H) is substituted with an amino acid group.

In Formula 1, A is a 5-membered to 10-membered heterocyclic group including one or two heteroatoms selected from the group consisting of oxygen and nitrogen, or a linear or branched C₁-C₃ alkylene group. The heterocyclic group may be unsubstituted or substituted with a hydroxyl group, a halogen atom, a cyano group, —C(═O)Rₐ, —C(═O)ORₐ, —OCO(ORₐ), —C═N(Rₐ), —SRₐ, —S(═O)Rₐ, —S(═O)₂Rₐ, —PRₐ, a substituted or unsubstituted C₁-C₂₀ alkyl group, a substituted or unsubstituted C₁-C₂₀ alkoxy group, a substituted or unsubstituted C₂-C₂₀ alkenyl group, a substituted or unsubstituted C₂-C₂₀ alkynyl group, a C₂-C₂₀ alkylene oxide group, a substituted or unsubstituted C₃-C₃₀ cycloalkyl group, a substituted or unsubstituted C₆-C₃₀ aryl group, a substituted or unsubstituted C₆-C₃₀ aryloxy group, a substituted or unsubstituted C₆-C₃₀ heteroaryl group, or any combination thereof. In this regard, Rₐ may be a hydrogen atom, a C₁-C₆ alkyl group, a C₂-C₆ alkenyl group, or a C₂-C₆ alkynyl group. The heterocyclic group may be a pyrimidine group or an isoxazole group. For example the heterocyclic group may be substituted with a methyl group or a methoxy group. The alkylene group may be unsubstituted or substituted with an amine group. R₁ may be a hydrogen atom, a hydroxyl group, an amine group, a ketone group, a halogen atom, a substituted or unsubstituted C₁-C₂₀ alkyl group, a cyano group, or any combination thereof. For example, R₁ may be an amine group.

The compound represented by Formula 1 may be sulfadiazine, sulfamethoxazole, sulfadoxine, or sulfaguanidine.

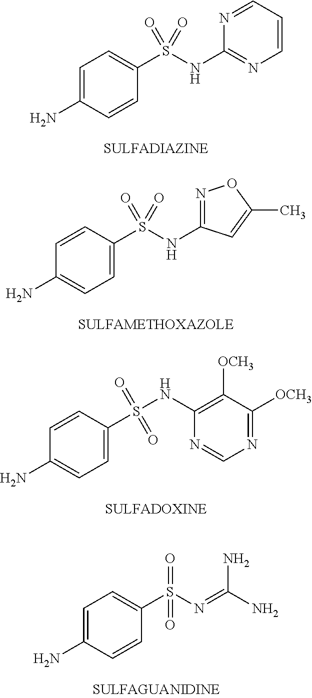 [SMILES: Nc1ccc(S(=O)(=O)Nc2ncccn2)cc1 ; Cc1cc(NS(=O)(=O)c2ccc(N)cc2)no1 ; COc1ncnc(NS(=O)(=O)c2ccc(N)cc2)c1OC ; NC(N)=NS(=O)(=O)c1ccc(N)cc1]

The compound represented by Formula 1 may be purchased from commercially available products, directly synthesized, or extracted, separated, or filtered from natural substances.

The pharmaceutically acceptable salt refers to a salt of a compound that does not cause significant irritation to an organism to which the salt is administered and does not damage the biological activity and physical properties of the compound. For example, the salt may be an inorganic acid salt, an organic acid salt, or a metal salt. Examples of the inorganic acid salt include hydrochloride, bromate, phosphate, sulfate, and disulfate. Examples of the organic acid salt include formate, acetate, propionate, lactate, oxalate, tartrate, malate, maleate, citrate, fumarate, besylate, camsylate, edisylate, trichloroacetate, trifluoroacetate, benzoate, gluconate, methane sulfonate, glycolate, succinate, 4-toluene sulfonate, galacturonate, embonate, glutamate, ethane sulfonate, benzene sulfonate, p-toluene sulfonate, or aspartate. Examples of the metal salt include calcium salt, sodium salt, magnesium salt, strontium salt, or potassium salt.

The solvate may be a compound produced by intermolecular attraction between a solute and a solvent. The solvate may be a hydrate.

The polymorph may be a substance present in more than one form or crystal structure.

The subject may be a mammal, such as a human, a cow, a horse, a pig, a dog, a sheep, a goat, or a cat. The subject may be a subject having muscular atrophy as a side effect of glucocorticoid treatment or glucocorticoid overproduction (e.g., elevated glucocorticoid levels as compared to a non-diseased subject of the same type not undergoing glucocorticoid treatment, whether due to increased production or decreased clearance of glucocorticoid from the body). Glucocorticoid overproduction can be caused by certain diseases (e.g., Cushing's syndrome).

The muscular atrophy may be caused by central nervous system damage, peripheral nerve damage, or a lesion of any portion of muscles and have various symptoms. The muscular atrophy may include diseases or states accompanied by loss of muscle strength accompanied by muscular atrophy, particularly, decreased muscle weight and loss of muscle strength of proximal muscles, decreased muscle function, loss of muscle mass, and the like. The loss of muscle may be caused by various factors such as genetic factors; age-related diseases such as hypertension, impaired glucose tolerance, diabetes, obesity, dyslipidemia, atherosclerosis, and cardiovascular diseases; chronic diseases such as cancer, autoimmune diseases, infectious diseases, acquired immunodeficiency syndrome (AIDS), chronic inflammatory diseases, arthritis, dystrophy, renal diseases, chronic obstructive pulmonary diseases, emphysema, rachitis, chronic lower back pain, peripheral nerve damage, central nervous system damage, and chemical damage; long-term fixed posture, fracture or trauma, and bed recovery from surgery; and progressive decrease in skeletal muscle mass and muscle strength associated with aging. The muscular atrophy may include amyotrophic lateral sclerosis, spinal progressive muscular atrophy, muscular dystrophy, or any combination thereof.

The muscular atrophy may be caused by side effects of glucocorticoid, for instance, due to glucocorticoid overproduction or glucocorticoid treatment. The term “glucocorticoid (GC)” refers to a steroid hormone binding to a glucocorticoid receptor (GR) or (GCR). The glucocorticoid may include at least one selected from the group consisting of cortisol, hydrocortine, cortisone, prednisolone, methylprednisolone, triamcinolone, triamcinolone acetonide, paramethasone, dexamethasone, betamethasone, hexestrol, methimazole, fluocinonide, fluocinolone acetonide, fluorometholone, beclomethasone dipropionate, estriol, diflorasone diacetate, diflucortolone valerate, and difluprednate.

Without wishing to be bound by any particular theory or mechanism of action, it is believed the muscular atrophy side effect of glucocorticoid treatment may be caused by an increase in Atrogin-1 activity as compared to Atrogin-1 activity levels in a subject not receiving glucocortocoid treatment, an increase in activity of muscle ring-finger protein-1 (MuRF-1) as compared to MuRF-1 activity levels in a subject not receiving glucocortocoid treatment, or any combination thereof. Atrogin-1, so called F-box only protein 32 (Fbox-32), is an F-box protein family encoded by an Fbox-32 gene in humans. The Atrogin-1 activity is an ability of constituting an ubiquitin protein ligase complex or an activity of the ubiquitin protein ligase complex. The Atrogin-1 may have NP_001229392 (human) and NP_080622 (mouse) amino acid sequences. In addition, the Atrogin-1 may be encoded by NM_001242463 (human) and NM_026346 (mouse) nucleotide sequences.MuRF-1, also called E3 ubiquitin-protein ligase TRIM63, is an enzyme encoded by a TRIM63 gene in humans. The MuRF-1 activity may be an activity of the E3 ubiquitin-protein ligase. The MuRF-1 may have NP_115977 (human) and NP_001034137 (mouse) amino acid sequences. In addition, the MuRF-1 may be encoded by NM_032588 (human) and NM_001039048 (mouse) nucleotide sequences. The term “increase in activity” refers to an increase in protein biosynthesis, an increase in specific activity of protein, or any combination thereof as compared to levels of protein biosynthesis or a protein's specific activity in a normal, non-diseased subject that does not have glucocorticoid overproduction and is not receiving glucocortocoid treatment.

The compound represented by Formula 1, a pharmaceutically acceptable salt, solvate, or polymorph thereof, or a combination of at least two of the foregoing may promote proliferation of skeletal muscle satellite cells, reduce the activity of Atrogin-1, reduce the activity of MuRF-1, or any combination thereof. The skeletal muscle satellite cells, also called satellite cells, refer to spindle-shaped mononuclear cells located between skeletal muscle fibers and basal lamina. When muscles are damaged, satellite cells may divide to regenerate muscles. The promoting of proliferation may be a decrease in doubling time of cell division, an increase in the number of cell divisions, or any combination thereof. The term “decrease in activity” refers to a decrease in protein biosynthesis, a decrease in specific activity of protein, or a combination thereof as compared to levels of protein biosynthesis or a protein's specific activity in a subject not receiving treatment with the compound represented by Formula 1. Thus, the compound represented by Formula 1, a pharmaceutically acceptable salt, solvate, or polymorph thereof, or a combination of at least two of the foregoing may prevent or treat muscular atrophy by promoting proliferation of skeletal muscle satellite cells and inhibiting muscle loss.

The term “preventing” refers to inhibiting or regarding side effects of glucocorticoid or the occurrence of diseases caused thereby by administering a composition. The term “treating” refers to relieving or alleviating side effects of glucocorticoid and diseases caused thereby by administering the composition.

The pharmaceutical composition may include the compound represented by Formula 1, a pharmaceutically acceptable salt, solvate, or polymorph thereof, or a combination of at least two of the foregoing in an effective amount. The effective amount may vary according to cells or subjects selected by those of ordinary skill in the art. The effective amount may be determined in accordance with side effects of glucocorticoid or seriousness of diseases caused thereby, age, weight, health, gender, and sensitivity to drugs of patients, administration time, administration route, and excretion rate of the patient, treatment time period, ingredients including drugs combined with or simultaneously used with the composition according to an exemplary embodiment, and other ingredients that are well known in medicines. The compound represented by Formula 1, a pharmaceutically acceptable salt, solvate, or polymorph thereof, or a combination of at least two of the foregoing may be included in a range of about 10 mg to about 10 g, about 50 mg to about 9 g, about 100 mg to about 8 g, about 200 mg to about 7 g, about 300 mg to about 6 g, about 400 mg to about 5 g, about 500 mg to about 4 g, about 500 mg to about 3 g, about 500 mg to about 2 g, or about 500 mg to about 1 g per the composition.

The pharmaceutical composition may be administered in a dose of about 0.001 mg/kg to about 100 mg/kg, 0.01 mg/kg to about 10 mg/kg, or 0.1 mg/kg to about 1 mg/kg of bodyweight for adults once a day, multiple times a day, or once every few days.

The pharmaceutical composition may further include a pharmaceutically acceptable additive. The pharmaceutical composition may be formulated into oral formulations or parenteral formulations. The oral formulations may be granules, powders, liquids, tablets, capsules, dry syrups, or any combination thereof. The parenteral formulations may be injections, or formulations for external use. Examples of the formulations for external use include cream, gel, ointment, skin emulsions, skin suspensions, transdermal patch, medication-containing bandage, lotions, or any combination thereof.

Also provided is a method of preventing or treating muscular atrophy in a subject includes administering the compound represented by Formula 1, a pharmaceutically acceptable salt, solvate, or polymorph thereof, or a combination of at least two of the foregoing to the subject.

The compound represented by Formula 1, the pharmaceutically acceptable salt, solvate, or polymorph thereof, the subject, the muscular atrophy, the preventing, and the treating are as described above.

The administration may be performed in a dose of about 0.001 mg/kg of bodyweight to about 100 mg/kg of bodyweight for adults once a day, multiple times a day, or once every few days for one day to one year. The administration may be performed using any known method. The administration may be performed directly to the subject via oral, intravenous, intramuscular, transdermal, mucosal, intranasal, intratracheal, or subcutaneous administration. The administration may be systemic or local administration.

Further provided is a method of promoting muscular regeneration in a subject includes administering the compound represented by Formula 1, a pharmaceutically acceptable salt, solvate, or polymorph thereof, or a combination of at least two of the foregoing into to subject.

The compound represented by Formula 1, the pharmaceutically acceptable salt, solvate, or polymorph thereof, the subject, and the administering are as described above.

The compound represented by Formula 1, a pharmaceutically acceptable salt, solvate, or polymorph thereof, or a combination of at least two of the foregoing may promote regeneration of muscular tissue by promoting proliferation of skeletal muscle satellite cells, thereby increasing muscle mass.

According to the methods of preventing or treating muscular atrophy or promoting muscular regeneration by using the compositions including the compound represented by Formula 1, a pharmaceutically acceptable salt, solvate, or polymorph thereof, or a combination of at least two of the foregoing, muscular atrophy may be efficiently prevented or treated or muscular regeneration may be efficiently promoted by promoting proliferation of skeletal muscle satellite cells and inhibiting degradation of muscular proteins.

Hereinafter, the following examples will be described in detail. These examples are not intended to limit the purpose and scope of the one or more embodiments of the inventive concept.

### EXAMPLE 1

### Identification of Preventive or Therapeutic Effects of Sulfonamide Compound on Muscular Atrophy

(1) Proliferation of Human Skeletal Muscle Satellite Cell by Sulfonamide Compound

Human skeletal muscle satellite (HSkMS) cells were prepared to identify whether proliferation of skeletal muscle satellite cells is promoted by sulfonamide compounds.

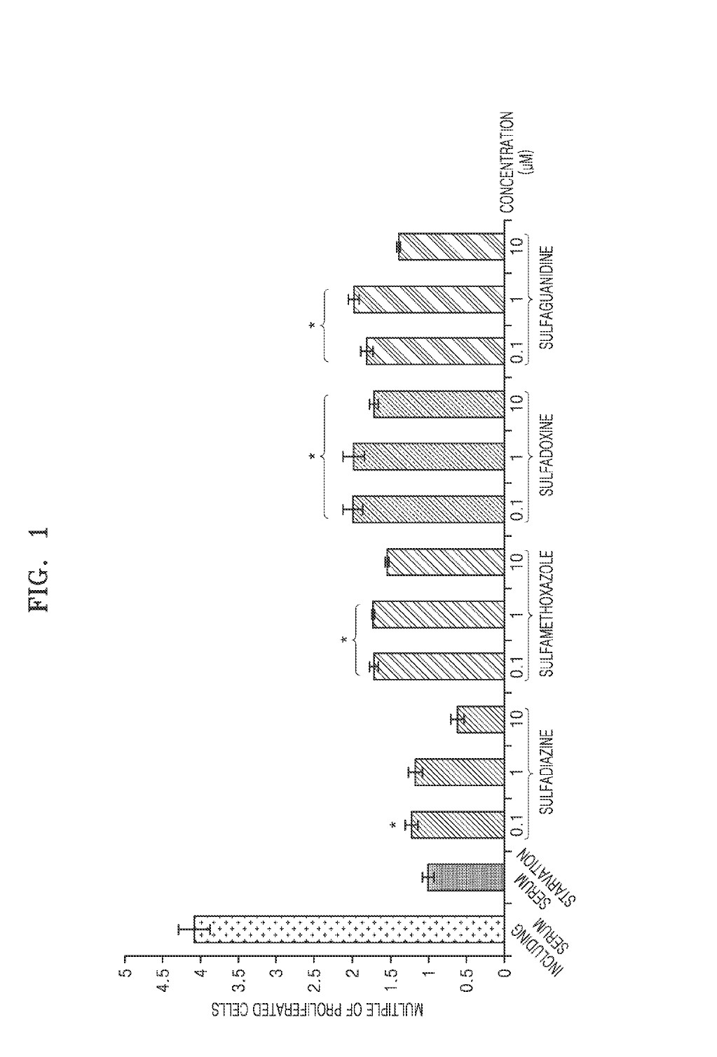

HSkMS cells (Innoprot) were seeded on a Skeletal Muscle Cell Medium (SkMCM), which does not include serum and a stem cell growth factor. Since the SkMCM does not include serum and a stem cell growth factor, the HSkMS cells may be induced into a state of serum starvation and growth factor starvation. The seeded HSkMS cells were cultured at 37° C. in a 5% CO₂ incubator for 24 hours. Then, 0.1 μM, 1 μM, and 10 μM of each of sulfadiazine (Sigma-Aldrich), sulfamethoxazole (Sigma-Aldrich), sulfadoxine (Sigma-Aldrich), and sulfaguanidine (Sigma-Aldrich), as sulfonamide compounds, were added to the HSkMS cells, and the cells were incubated at 37° C. in a 5% CO₂ incubator for 24 hours. After the incubation, the numbers of HSkMS cells were calculated using a hemocytometer. The numbers of HSkMS cells are shown in FIG. 1 as multiples of the number of HSkMS cells of a negative control group to which the sulfonamide compound was not added (*: p<0.05).

As illustrated in FIG. 1, the proliferation of the HSkMS cells was promoted in all groups, in comparison with serum starvation, except for the groups using 10 μM of sulfadiazine, sulfamethoxazole or sulfaguanidine, or 1 μM of sulfadiazine. Particularly, it was confirmed that muscular regeneration may be promoted by using low concentrations of a sulfonamide compound since proliferation of the HSkMS cells was promoted by using 0.1 μM and 1 μM of several sulfonamide compounds.

(2) Effect of Sulfadiazine on Inhibition of Activity of Muscle Protease

C2C12 cell lines (mouse gastrocnemius muscle, ATCC® CRL-1772™) were seeded in a 10% FBS/DMEM and incubated at 37° C. in a 5% CO₂ incubator. When the cells were 70% confluent, the culture medium was replaced with 2% FBS/DMEM and the cells were cultured for 4 days to completely induce differentiation into muscle cells. C2C12 cell lines are mouse myoblast cell lines obtained by continuous culture of myoblast cells cultured from gastrocnemius muscle of C3H mice.

When C2C12 cells were completely differentiated into muscle cells, the culture medium was removed therefrom, and 4 ml of a fresh culture medium including 1 μM, 10 μM, or 100 μM of sulfadiazine (Sigma Aldrich) was added thereto, and then, the cells were cultured at 37° C. in a 5% CO₂ incubator for 24 hours. 2 μM of dexamethasone (Sigma Aldrich) was added to the cultured cells and the cells were further cultured for 24 hours. A negative control group was not treated with sulfadiazine and dexamethasone.

A Trizol reagent (Invitrogen) was added to the collected cells, and total RNA was separated therefrom according to manufacturer's protocols. Complementary DNA (cDNA) was synthesized from the separated total RNA by using a reverse transcriptase. Amounts of mRNA of Atrogin-1 and MuRF-1 were quantified using a real time PCR device (MyiQPCR instrument, BioRad) that is a device measuring SYBR Green. The results were normalized with respect to an amount of GAPDH mRNA. Amplification of Atrogin-1, MuRF-1, and GAPDH mRNA was performed using an oligonucleotide set of SEQ ID NOs: 1 and 2, an oligonucleotide set of SEQ ID NOs: 3 and 4, and an oligonucleotide set of SEQ ID NOs: 5 and 6 as primers, respectively.

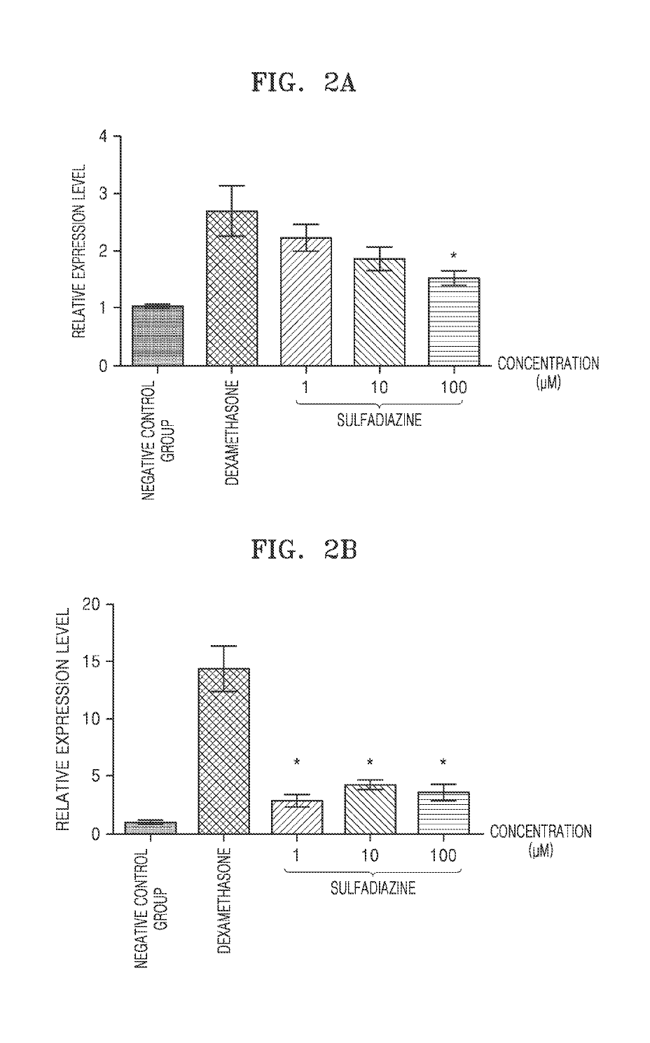

Values obtained via amplification were normalized to GAPDH gene expression, and relative expression levels of mRNA of Atrogin-1 and MURF-1 were quantified. The quantified relative expression levels (arbitrary unit (AU)) of mRNA of Atrogin-1 and MURF-1 are illustrated in FIGS. 2A and 2B, respectively (*: p<0.05 vs. dexamethasone)

As illustrated in FIGS. 2A and 2B, while the expression level of Atrogin-1 and MuRF1 increased in cells treated with only dexamethasone, the expression of mRNA of Atrogin-1 and MuRF1 decreased in cells treated with both sulfadiazine and dexamethasone. Thus, it was confirmed that the sulfonamide compound including sulfadiazine inhibited the expression of Atrogin-1 and MuRF1, which are components of ubiquitin ligase complex in ubiquitin-mediated protein degradation pathway thereby causing muscle loss, and thus, the degradation of muscle fiber caused by dexamethasone was inhibited, thereby preventing or treating muscle loss.

(3) Effects of Sulfadiazine on Protection of Muscle Fiber

Diameters of muscle fibers according to the addition of dexamethasone and/or sulfadiazine were measured to identify the effects of sulfonamide compounds including sulfadiazine on the protection of muscle fiber.

As described above with reference to Example 1(2), C2C12 cells were completely differentiated into muscle cells. After removing the culture medium, 4 ml of a fresh culture medium including 100 μM of sulfadiazine (Sigma Aldrich) was added thereto, and then, the cells were cultured at 37° C. in a 5% CO₂ incubator for 24 hours. 2 μM of dexamethasone (Sigma Aldrich) was added to the cultured cells, and the cells were further cultured for 24 hours. A negative control group was not treated with sulfadiazine and dexamethasone.

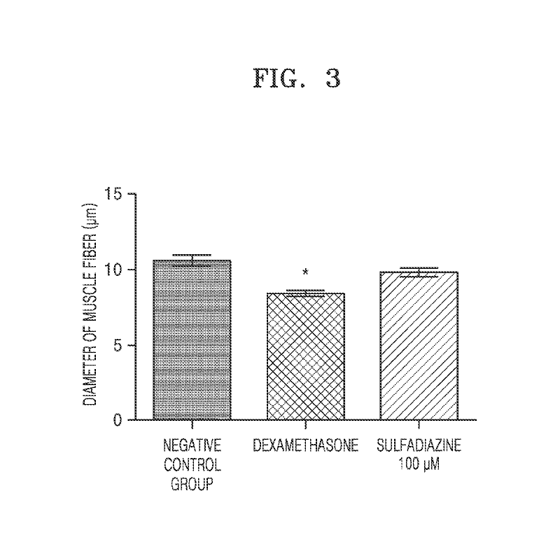

A diameter (μm) of a muscle fiber was measured from a microscopic image (X400) of the muscle fiber of the cells, and the results are shown in FIG. 3 (*: p<0.05).

As illustrated in FIG. 3, in a group treated with only dexamethasone but not with sulfadiazine, muscular atrophy was observed since the diameter of the muscle fiber was reduced. However, the diameter of the muscle fiber of a group pre-treated with sulfadiazine was similar to that of the control group. Thus, it was confirmed that the sulfonamide compound including sulfadiazine may inhibit loss of muscle mass.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of preventing or treating muscular atrophy in a subject, the method comprising administering to the subject a compound of Formula 1, or pharmaceutically acceptable salt, solvate, or polymorph thereof, or a combination thereof: 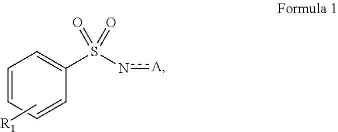
wherein the dotted line is an optional double bond,
wherein A is a 5-membered to 10-membered heterocyclic group including one or two heteroatoms selected from the group consisting of oxygen and nitrogen, or a linear or branched C₁-C₃ alkyl group,
wherein the heterocyclic group is unsubstituted or substituted with a hydroxyl group, a halogen atom, a cyano group, —C(═O)Rₐ, —C(═O)ORₐ, —OCO(ORₐ), —C═N(Rₐ), —SRₐ, —S(═O)Rₐ, —S(═O)₂Rₐ, —PRₐ, a substituted or unsubstituted C₁-C₂₀ alkyl group, a substituted or unsubstituted C₁-C₂₀ alkoxy group, a substituted or unsubstituted C₂-C₂₀ alkenyl group, a substituted or unsubstituted C₂-C₂₀ alkynyl group, a C₂-C₂₀ alkylene oxide group, a substituted or unsubstituted C₃-C₃₀ cycloalkyl group, a substituted or unsubstituted C₆-C₃₀ aryl group, a substituted or unsubstituted C₆-C₃₀ aryloxy group, a substituted or unsubstituted C₆-C₃₀ heteroaryl group, or any combination thereof,
and Rₐ is a hydrogen atom, a C₁-C₆ alkyl group, a C₂-C₆ alkenyl group, or a C₂-C₆ alkynyl group,
and wherein the alkyl group is unsubstituted or substituted with one or more amine groups, and
R₁ is a hydrogen atom, a hydroxyl group, an amine group, a ketone group, a halogen atom, a substituted or unsubstituted C₁-C₂₀ alkyl group, a cyano group, or any combination thereof.

2. The method of claim 1, wherein the heterocyclic group is a pyrimidine group or an isoxazole group.

3. The method of claim 1, wherein the compound represented by Formula 1 is sulfadiazine, sulfamethoxazole, sulfadoxine, or sulfaguanidine.

4. The method of claim 1, wherein the muscular atrophy is caused by side effects of glucocorticoid.

5. The method of claim 1, wherein the subject has muscular atrophy as a side effect of a glucocorticoid.

6. The method of claim 5, wherein the muscular atrophy as a side effect of glucocorticoid is caused by an increase in Atrogin-1 activity, an increase in MuRF-1 activity, or both, as compared to Atrogin-1 or MuRF-1 activity levels in a normal, non-diseased subject.

7. The method of claim 1, wherein the administering promotes proliferation of skeletal muscle satellite cells, reduces Atrogin-1 activity, reduces MuRF-1 activity, or achieves any combination thereof.

8. The method of claim 1, wherein the subject has amyotrophic lateral sclerosis, spinal progressive muscular atrophy, muscular dystrophy, or any combination thereof.

9. A method of promoting regeneration of muscle tissue in a subject, the method comprising administering to the subject the compound of Formula 1, a pharmaceutically acceptable salt, solvate, or polymorph thereof, or a combination thereof: 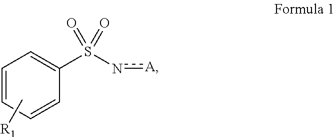
wherein the dotted line is an optional double bond,
wherein A is a 5-membered to 10-membered heterocyclic group including one or two heteroatoms selected from the group consisting of oxygen and nitrogen, or a linear or branched C₁-C₃ alkyl group,
wherein the heterocyclic group is unsubstituted or substituted with a hydroxyl group, a halogen atom, a cyano group, —C(═O)Rₐ, —C(═O)ORₐ, —OCO(ORₐ), —C═N(Rₐ), —SRₐ, —S(═O)Rₐ, —S(═O)₂Rₐ, —PRₐ, a substituted or unsubstituted C₁-C₂₀ alkyl group, a substituted or unsubstituted C₁-C₂₀ alkoxy group, a substituted or unsubstituted C₂-C₂₀ alkenyl group, a substituted or unsubstituted C₂-C₂₀ alkynyl group, a C₂-C₂₀ alkylene oxide group, a substituted or unsubstituted C₃-C₃₀ cycloalkyl group, a substituted or unsubstituted C₆-C₃₀ aryl group, a substituted or unsubstituted C₆-C₃₀ aryloxy group, a substituted or unsubstituted C₆-C₃₀ heteroaryl group, or any combination thereof, and Rₐ is a hydrogen atom, a C₁-C₆ alkyl group, a C₂-C₆ alkenyl group, or a C₂-C₆ alkynyl group,
and wherein the alkyl group is unsubstituted or substituted with one or more amine groups, and
R₁ is a hydrogen atom, a hydroxyl group, an amine group, a ketone group, a halogen atom, a substituted or unsubstituted C₁-C₂₀ alkyl group, a cyano group, or any combination thereof.

10. The method of claim 9, wherein the heterocyclic group is a pyrimidine group or an isoxazole group.

11. The method of claim 9 wherein the compound represented by Formula 1 is sulfadiazine, sulfamethoxazole, sulfadoxine, or sulfaguanidine.

12. The method of claim 9, wherein the administering promotes proliferation of skeletal muscle satellite cells.

13. The method of claim 1, wherein the compound of Formula 1, the pharmaceutically acceptable salt, solvate, or polymorph thereof, or the combination thereof, is administered in a dose of about 0.001 mg/kg to about 100 mg/kg.

14. The method of claim 1, wherein the compound of Formula 1, the pharmaceutically acceptable salt, solvate, or polymorph thereof, or the combination thereof, is administered via oral, intravenous, intramuscular, transdermal, mucosal, intranasal, intratracheal, or subcutaneous administration.

15. The method of claim 1, wherein the compound of Formula 1, the pharmaceutically acceptable salt, solvate, or polymorph thereof, or the combination thereof is administered once a day, multiple times a day, or once every second day to once a year.

16. The method of claim 9, wherein the compound of Formula 1, the pharmaceutically acceptable salt, solvate, or polymorph thereof, or the combination thereof, is administered in a dose of about 0.001 mg/kg to about 100 mg/kg.

17. The method of claim 9, wherein the compound of Formula 1, the pharmaceutically acceptable salt, solvate, or polymorph thereof, or the combination thereof, is administered via oral, intravenous, intramuscular, transdermal, mucosal, intranasal, intratracheal, or subcutaneous administration.

18. The method of claim 9, wherein the compound of Formula 1, the pharmaceutically acceptable salt, solvate, or polymorph thereof, or the combination thereof is administered once a day, multiple times a day, or once every second day to once a year.


SEQUENCE LISTING (6 sequences)

SEQ ID NO: 1 - DNA, 21 bp, Artificial Sequence
  primer for mouse atrogin-1/MAFbx
cacattctctcctggaagggc

SEQ ID NO: 2 - DNA, 21 bp, Artificial Sequence
  primer for mouse atrogin-1/MAFbx
ttgataaagtcttgaggggaa

SEQ ID NO: 3 - DNA, 20 bp, Artificial Sequence
  primer for mouse MuRF-1
acgagaagaagagcgagctg

SEQ ID NO: 4 - DNA, 22 bp, Artificial Sequence
  primer for mouse MuRF-1
cttggcacttgagagaggaagg

SEQ ID NO: 5 - DNA, 20 bp, Artificial Sequence
  primer for mouse GAPDH
catggccttccgtgttccta

SEQ ID NO: 6 - DNA, 17 bp, Artificial Sequence
  primer for mouse GAPDH
gcggcacgtcagatcca